(19) United States
(12) Patent Application Publication
(10) Pub. No.: US 2008/0015452 A1
(43) Pub. Date: Jan. 17, 2008

(54) Method of processing electrocardiogram waveform

(76) Inventors: Carlos A. Ricci, Apple Valley, MN (US); Vladimir V. Kovtun, Inver Grove Heights, MN (US)

Correspondence Address: DICKE, BILLIG & CZAJA, FIFTH STREET TOWERS, 100 SOUTH FIFTH STREET, SUITE 2250, MINNEAPOLIS, MN, 55402, US

(21) Appl. No.: 11/479,674

(22) Filed: Jun. 30, 2006

(51) Int. Cl. A61B 5/04

(52) U.S. Cl. 600/509

(57) ABSTRACT

A method of analyzing an elecotrocardiogram (ECG) waveform, the method including obtaining a series of digital values representing the ECG waveform having a plurality of cycles, with each cycle having a T-wave feature. The method includes comparing a first prototype wavelet to a portion of the series of digital values representing a selected number of cycles of the ECG waveform, wherein the first prototype wavelet is defined by a first set of wavelet parameters having an initial set of first parameter values, and adjusting the initial set of first parameter values to obtain a matched prototype wavelet defined by the first set of wavelet parameters having a matched set of first parameter values for each of the T-wave features of the selected number of cycles, wherein each matched prototype wavelet substantially matches the corresponding T-wave feature.

### BACKGROUND

The digital representation and analysis of waveforms for detection of periodic and non-periodic features is central to various sectors of industry for determining the existence of certain conditions. For example, in the field of cardiology, many aspects of the physical condition of the human heart are reflected in an electrocardiogram (ECG) waveform. Analysis of electrical cardiac activity can provide significant insights into the risk state of a patient for sudden cardiac death (SCD). Identification of spurious electrical activity within the heart can provide a physician with clues as to the relative cardiac risk presented to the patient.

For instance, analysis of T-wave alternans is one method employed for identifying risk of sudden cardiac death. As generally defined, T-wave alternans refers to an alternation in the morphology of the T-wave in an AB-AB beat pattern. In particular, different rates of repolarization of the muscle cells in the ventricles of the heart in an alternating beat-by-beat pattern have been associated with a variety of clinical conditions including prolonged QT syndrome, acute myocardial ischemia, and electrolyte disturbance. Nonuniform repolarization is associated with electrical instability in the heart. T-wave alternans has been recognized as a significant indicator of risk for ventricular arrhythmia and (SCD).

Visual analysis of T-wave alternans using an ECG is typically impractical due to the minute differences in signal amplitude of T-waves between alternating beats relative to other variations in the ECG resulting from respiration components and noise, for example. However, T-wave alternans at a microvolt level has been identified as an indicator of electrically unstable myocardium. As such, several computer-based morphology analysis techniques have been developed for T-wave analysis.

One such technique involves calculating A and B modified moving averages for alternating ECG beats. The T-wave alternans estimate for an ECG segment is determined as the maximum absolute difference between the A and B modified moving averages computed over the ST segment and T-wave regions of the ECG. Such an approach provides only an amplitude estimate of T-wave alternans.

Additionally, for mobile telecardiology applications and to lessen data storage and transmission requirements for the large volume of ECG data generated for patient monitoring and diagnosis, an ECG analysis technique which enables ECG data compression without degrading the value of waveform data is also desired.

### SUMMARY

One embodiment provides a method of analyzing an elecotrocardiogram (ECG) waveform, the method including obtaining a series of digital values representing the ECG waveform having a plurality of cycles, with each cycle having a T-wave feature. The method includes comparing a first prototype wavelet to a portion of the series of digital values representing a selected number of cycles of the ECG waveform, wherein the first prototype wavelet is defined by a first set of wavelet parameters having an initial set of first parameter values, and adjusting the initial set of first parameter values to obtain a matched prototype wavelet defined by the first set of wavelet parameters having a matched set of first parameter values for each of the T-wave features of the selected number of cycles, wherein each matched prototype wavelet substantially matches the corresponding T-wave feature.

### BRIEF DESCRIPTION OF THE DRAWINGS

FIG. 1 is a block diagram illustrating generally one embodiment of signal analyzer employing wavelet matching techniques.

FIG. 2A is an example plot of an electrocardiogram.

FIG. 2B is an example plot illustrating features of an electrocardiogram.

FIG. 3 is a block diagram illustrating an example implementation of a Kovtun-Ricci wavelet.

FIG. 4 is a block diagram illustrating an example implementation of a Kovtun-Ricci wavelet.

FIG. 5 is a block diagram illustrating an example implementation of a Kovtun-Ricci wavelet.

FIG. 6 is a block diagram illustrating an example implementation of a Kovtun-Ricci wavelet.

FIG. 7 is a block diagram illustrating an example implementation of a Kovtun-Ricci wavelet.

FIG. 8 is a plot illustrating an example impulse response of a Kovtun-Ricci wavelet.

FIGS. 9A-9E are plots illustrating the effect of varying order parameter values on the impulse response wave shape of a Kovtun-Ricci wavelet.

FIG. 10 is a plot illustrating the effect of different scale parameters values on the impulse response wave shape of a Kovtun-Ricci wavelet.

FIG. 11 is a plot illustrating impulse response wave shapes of a real component and an imaginary component of a Kovtun-Ricci wavelet.

FIGS. 12A-12H are plots illustrating the effect of varying real and complex coefficient values on the impulse response wave shape of a Kovtun-Ricci wavelet.

FIG. 13 is a plot illustrating an example of an impulse response of a Kovtun-Ricci wavelet matched to a T-wave feature of an electrocardiogram.

FIG. 14 is a plot illustrating an example of a series of impulse response wave shapes of a Kovtun-Ricci wavelet matched to a series of T-wave features of an electrocardiogram.

FIG. 15 is a table illustrating T-wave parameters derived using a matched Kovtun-Ricci wavelet.

FIG. 16 is a flow diagram illustrating a process for matching a Kovtun-Ricci wavelet to a waveform feature.

FIG. 17 is a flow diagram illustrating a process for matching a Kovtun-Ricci wavelet to a waveform feature.

FIG. 18 is a plot illustrating an example of an impulse response wave shape matched to a T-wave feature and an impulse response wave shape matched to a QRS-complex of an electrocardiogram.

FIG. 19 is a table illustrating T-wave parameters derived using a Kovtun-Ricci wavelet.

FIG. 20 is a flow diagram illustrating a process for matching a Kovtun-Ricci wavelet to a waveform feature.

### DETAILED DESCRIPTION

In the following Detailed Description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments of the present invention can be positioned in a number of different orientations, the directional terminology is used for purposes of illustration and is in no way limiting. It is to be understood that other embodiments may be utilized and structural or logical changes may be made without departing from the scope of the present invention. The following detailed description, therefore, is not to be taken in a limiting sense, and the scope of the present invention is defined by the appended claims.

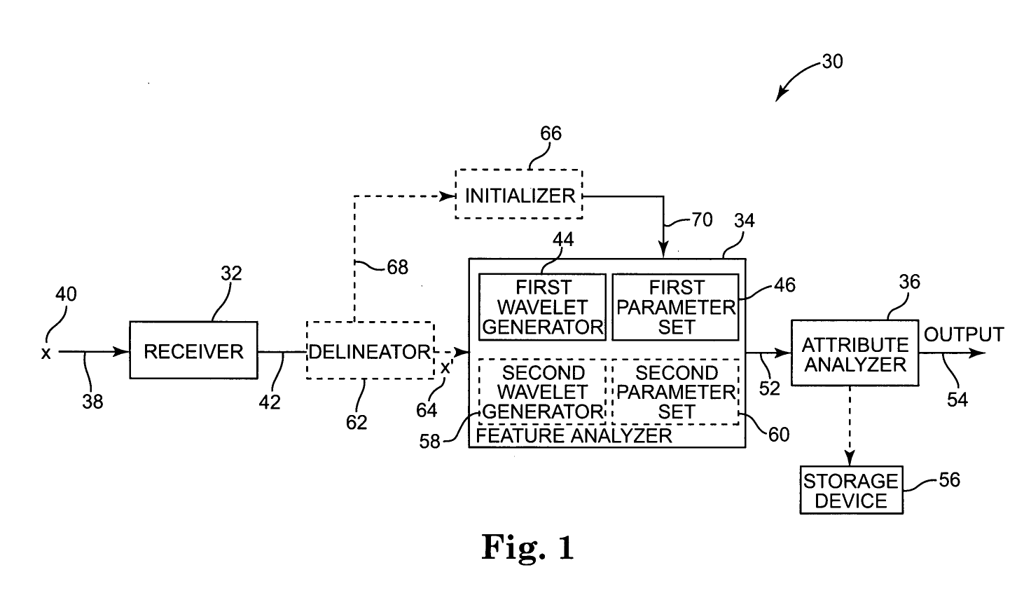

FIG. 1 is a block diagram illustrating generally one embodiment of signal analyzer 30 employing wavelet matching techniques such as the wavelet matching techniques described herein. Signal analyzer 30 includes a receiver 32, a feature analyzer 34, and an attribute analyzer 36. Receiver 32 receives at 38 an input signal x 40 comprising a series of digital values representative of a quasi-periodic (qp) waveform having a plurality of cycles. Quasi-periodic, as employed herein, is considered a signal or waveform having one or more locally periodic features having varying periods and/or shapes. Locally periodic, as used herein, means that a feature is present in at least one cycle (e.g., a sporadic transient feature), but is generally present on the order of several, but up to all, cycles. In one embodiment, input signal x 40 comprises a qp waveform having a plurality of cycles, with each cycle having at least a first feature. In one embodiment, as described in greater detail below, input signal x 40 comprises an electrocardiogram (ECG) signal.

In one embodiment, feature analyzer 34 receives the series of digital values from receiver 32 via a signal path 42. Feature analyzer 34 includes a first wavelet generator 44 configured to generate a family of prototype wavelets defined by a first set of wavelet parameters 46. The prototype wavelets are formed from any type of wavelet described by a finite set of parameters, including wavelets from the Haar, Morlet, Complex Morlet, Guassian, Complex Gaussian, Daubechies, Spline, Meyer, and Mexican Hat wavelet families, or wavelets derived from scales of the shape of the desired feature. (See Wavelets and Subband Coding, M. Vetterli and J. Kovacevic, Prentice-Hall, 1995.) In one embodiment, as described in greater detail below, first wavelet generator 44 is configured to generate a family of Kovtun-Ricci (KR) prototype wavelets.

In one embodiment, first wavelet generator 44 generates a first prototype wavelet defined by the first set of wavelet parameters 46 having an initial set of first parameter values. In one embodiment, as described in greater detail below, the prototype wavelets generated by first wavelet generator 44 include a real wavelet component and an imaginary wavelet component. In one embodiment, as described in greater detail below, the set of first wavelet parameters 46 includes at least one order parameter, at least one scale parameter, at least one amplitude coefficient parameter pair including a real amplitude coefficient parameter and an imaginary amplitude coefficient parameter, and at least one central index parameter.

In one embodiment, feature analyzer 34 compares the first prototype wavelet to at least a portion of a series of data values corresponding to a selected number of cycles of input signal x 40. In one embodiment, the selected number of cycles comprises all cycles of input signal x 40. In one embodiment, the selected number of cycles of input signal x 40 comprises cycles satisfying a predefined selection criterion. Feature analyzer 34 adjusts the initial set of first parameter values to obtain a matched prototype wavelet defined by the first set of wavelet parameters having an adjusted, or matched, set of first parameter values for each of the first features of the selected number of cycles, wherein each matched prototype wavelet substantially matches the corresponding first feature. In one embodiment, as will be described in greater detail below, feature analyzer 34 individually obtains a matched wavelet for each of the first features by iteratively adjusting the values of the set of first parameter values until an error metric between a prototype wavelet generated by first wavelet generator 44 and the corresponding first feature is substantially optimized. The values of the set of first parameter values when the error metric is substantially optimized comprises the matched set of first parameter values which define the matched wavelet.

In one embodiment, the error metric comprises a mean-square error or sum-square error between the prototype wavelet and the corresponding first feature. In another embodiment, the error metric comprises a measure of the maximum absolute deviation between the prototype wavelet and the corresponding first feature. In another embodiment, the error metric comprises a measure of the area of the absolute value of a difference between the prototype wavelet and the corresponding first feature. Optimization of the parameter values comprises a means to minimize the error metric and may include a local or global search over a vector space represented by the parameters. Other suitable parameter optimization techniques may also be employed such as, for example, line minimization, simulated annealing, Lagrange Multipliers, and Gauss-Newton methods. (See also Optimization by Vector Space Methods, by David G. Luenberger, John Wiley & Sons, Inc., 1969). In some embodiments, optimization methods may be applied separately, or in combination, to certain subspaces of the parametric space to enable optimization of efficiency of the error minimization algorithm.

Attribute analyzer 36 receives a stream of sets of matched first parameter values, one set of matched first parameter values for each of the first features, from feature analyzer 34 via a signal path 52. Attribute analyzer 36 provides an output signal 54 indicative of one or more attributes of the first features based on the stream of sets of matched first parameter values for the first set of wavelet parameters 46. In one embodiment, attribute analyzer 36 provides output signal 54 indicative of an absolute complex amplitude attribute for each of the first features based on the corresponding real and imaginary amplitude coefficient parameters. In one embodiment, attribute analyzer 36 provides output signal 54 indicative of a complex angle attribute for each of the first features based on the corresponding real and imaginary amplitude coefficient parameters. In one embodiment, attribute analyzer 36 provides output signal 54 indicative of timing relationships between first features based on their corresponding central index parameters.

In one embodiment, the providing of output signal 54 by attribute analyzer 36 includes transmitting the set of matched first parameter values (e.g., an order parameter value, a scale parameter value, a central index parameter value, and real and imaginary amplitude coefficient parameter values) and/or the corresponding feature attributes (e.g., absolute complex amplitude attribute and complex angle attribute) for each of the first features of the selected cycles of input signal x 40 to a remote receiving device (not shown).

In one embodiment, attribute analyzer 36 stores the set of matched first parameter values and/or the corresponding feature attributes and/or the matched wavelets for each of the first features of the selected cycles of input signal x 40 in a storage device 56. In one embodiment, the set of matched first parameter values for each of the first features are stored in storage device 56 in the form of individually identifiable wavelet parameter objects. For example, in one embodiment, each wavelet parameter object is indexed to a first feature of a particular cycle of input signal x 40, and includes an order parameter value, a scale parameter value, a central index parameter value, and a real and an imaginary amplitude coefficient parameter value. Such an object representation creates a compact packet of concentrated information about the first feature, and enables the first features of each of the plurality of cycles of input signal x 40 to be stored and represented as a series of wavelet parameter objects, in lieu of the corresponding individual digital data values of input signal x 40, thereby reducing data storage and transmission requirements.

In some embodiments, these wavelet parameter objects may be represented as structures or objects in a computer program. The representation of data through use of such objects in well known in the arts of software engineering and computer science. (See Data Structures and Other Objects Using C++ 2^{nd} Edition, by M. Main and W. Savitch, Addison-Wesley, 2001.) The use of the term object implies a meaning as understood by these arts.

Although described generally above in terms of a first feature, each cycle of quasi-periodic input signal x 40 may include more than one feature of interest or “target” feature. In one embodiment, in addition to the first feature, each cycle of input signal x 40 includes a second feature. As such, in one embodiment, feature analyzer 34 further includes a second wavelet generator 58 configured to generate a family of prototype wavelets defined by a second set of wavelet parameters 60. In one embodiment, second wavelet generator 58 generates a second prototype wavelet defined by the second set of wavelet parameters 60 having an initial set of second parameter values. In one embodiment, second wavelet generator 58 comprises the same wavelet generator as first wavelet generator 44 (e.g., Kovtun-Ricci prototype wavelet) with second set of wavelet parameters 60 comprising the same wavelet parameter types as first set of wavelet parameters 46. In a fashion similar to that described above with respect to first wavelet generator 44, feature analyzer 34 compares the second prototype wavelet to each cycle of the selected number of cycles of input signal x 40 and adjusts the initial set of second parameter values to obtain a matched prototype wavelet defined by the second set of wavelet parameters having an adjusted, or matched, set of second parameter values for each of the second features of the selected number of cycles, wherein each matched prototype wavelet substantially matches the corresponding second feature.

Likewise, attribute analyzer 36 provides output signal 54 indicative of one or more attributes of the second features (e.g., absolute complex amplitude attribute and complex angle attribute) based on the sets of matched second parameter values. In one embodiment, attribute analyzer 36 provides output signal 54 further indicative of one or more attributes relative to the first and second features, such as, for example, a time interval between first and second features of a same cycle based on the corresponding central index parameters. Similarly, in one embodiment, attribute analyzer 36 respectively stores and/or transmits the set of matched parameter values and/or the corresponding attributes for each of the second features of the selected cycles of input signal x 40 to storage device 56 or a remote receiving device (not shown).

By extension, in other embodiments, feature analyzer 34 includes a plurality of wavelet generators, each configured to generate a family of prototype wavelets defined by a corresponding set of wavelet parameters, and configured to obtain a matched prototype wavelet by adjusting the corresponding sets of parameter values to obtain sets of matched parameter values such that each of the matched prototype wavelets substantially matches a feature of a corresponding plurality of features of each cycle of the selected number of cycles of input signal x 40.

In one embodiment, signal analyzer 30 further includes a delineator 62. Delineator 62 receives input signal x 40 via signal path 42 and determines points of interest of the qp waveform represented by input signal x 40, wherein the points of interest are indicative of features of the qp waveform, such as the first feature, for example. Examples of such points of interest include local peaks, valleys, inflections, and changes of slope of the qp waveform.

In one embodiment, based on the determined points of interest, delineator 62 provides a segmented input signal x′ 64 to feature analyzer 34, wherein segmented input signal x′ 64 comprises only portions or segments of input signal x 40, wherein each segment comprises a range of data points corresponding to a selected feature of the qp waveform. In one embodiment, the selected feature comprises the first feature, such that each of the segments of segmented input signal x′ 64 comprises a range of data points of input signal x 40 which includes and corresponds to a first feature of the qp waveform represented by input signal x 40. For example, in one embodiment, as will be described in greater detail below, each segment of segmented signal x′ 64 comprises a range of data points corresponding to a T-wave feature of an ECG waveform represented by input signal x 40.

An example of one technique which can be employed by delineator 62 for determining feature points of and segmenting input signal x 40 is a quarter-phase transition technique described by U.S. patent application Ser. No. 11/360,223, filed on Feb. 23, 2006, entitled “System and Method for Signal Decomposition, Analysis, and Reconstruction” which is herein incorporated by reference. In other embodiments, delineator 62 employs conventional techniques generally known in the art such as QRS-complex detection, QT interval measurement, and J point estimation. In one embodiment, delineator 62 determines and provides segmented input signal x′ 64 based on known and/or expected characteristics of the qp waveform represented by input signal x 40.

In one embodiment, signal analyzer 30 further includes an initializer 66. In one embodiment, initializer 66 receives segmented input signal x′ 64 and information representative of attributes (e.g., position, amplitude) of the points of interest along input signal x 40 via a signal path 68. In one embodiment, based on the information received from initializer 66, initializer 66 determines and provides, via a signal path 70, initial parameter values for one or more of the parameters of the first set of wavelet parameters 46. In one embodiment, initializer 66 determines and provides initial parameter values for one or more of the parameters of the first set of wavelet parameters 46 based on the information received via signal path 68 and on predetermined knowledge of input signal x 40 (e.g., general characteristics of an ECG).

In one embodiment, as mentioned above, the information provided by delineator 62 to initializer 66 via signal path 68 is based upon quarter-phase analysis as described by the previously incorporated U.S. patent application Ser. No. 11/360,223. For example, an atomic period may be used as an initial value estimate for the scale parameter of the first set of wavelet parameters 46. Also, sequence detection of a phase label sequence arising from a corresponding component set may be employed to provide an initial estimate for the central index parameter of the first set of wavelet parameters 46. Additionally, an estimate of the order parameter may be determined based on a width of an amplitude envelope surrounding the estimated central index parameter.

By providing initial parameter values and segmented input signal x′ 64 to feature analyzer 34, feature analyzer 34 is able to improve the efficiency and, thus, reduce a time required to perform the typically iterative optimization process for determining the sets of matched parameter values which produce the matched wavelets.

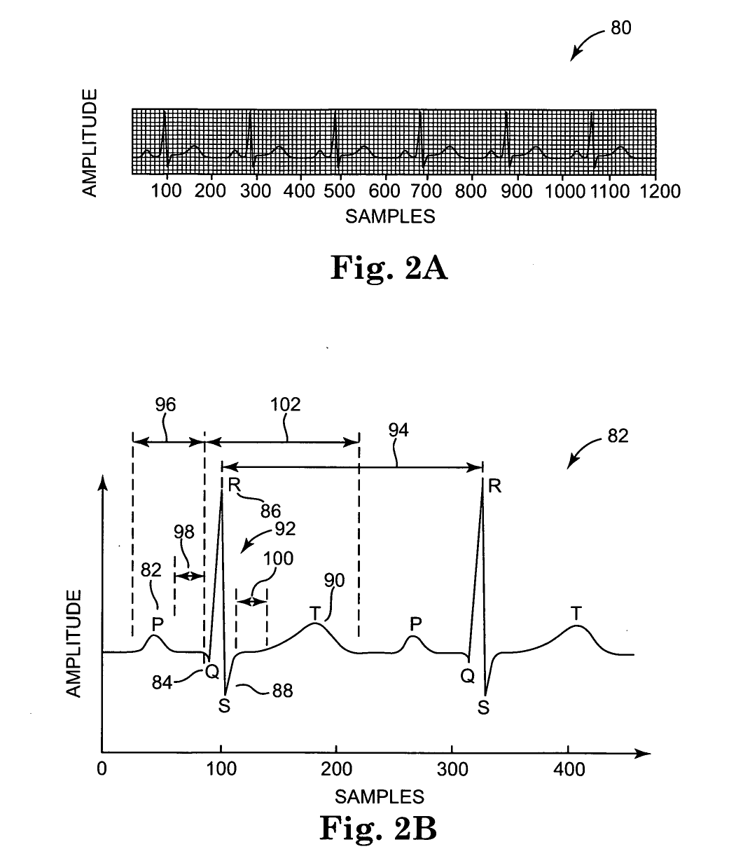

In one embodiment, as described briefly above, input signal x 40 is representative of an ECG waveform, which is a measurement of the electrical activity of the heart. FIG. 2A is a graph 80 representative of a human surface ECG signal. FIG. 2B is a graph 82 illustrating in greater detail a portion of an ECG signal and illustrating generally the typical features of an ECG waveform. As illustrated, the ECG waveform includes a P-wave 82, a Q-wave 84, an R-wave 86, an S-wave 88, and a T-wave 90. P-wave 82 is caused by excitation of the atria. Together, Q-wave 84, R-wave 86, and S-wave 88 form what is commonly referred to as the QRS-complex, indicated at 92, which results from the excitation (de-polarization) of the ventricles. T-wave 90 results from recovery (re-polarization) of the ventricles. The interval between the apexes of consecutive R-waves, referred to as the R-R interval 94, corresponds to one cycle of the ECG, sometimes referred to as a cardiac cycle or heart beat.

A portion of the ECG from a beginning of P-wave 82 (onset of atrial de-polarization) to a beginning of QRS-complex 92 is referred to as the PR-interval, as indicated at 96. A portion from an end of P-wave 82 to the beginning of QRS-complex 92 is referred to as the PR-segment, as indicated at 98. PR-segment 98 corresponds to the time between the end of the atrial de-polarization to the onset of ventricular de-polarization. QRS-complex 92 represents the time required for de-polarization of the ventricles. A portion of the ECG between S-wave 88 and T-wave 90 is known as the ST-segment, as indicated at 100. ST-segment 100 corresponds to a period of ventricular muscle activity before re-polarization. A portion from an onset of QRS-complex 92 to an end of T-wave 90 is known as the QT-interval, as indicated at 102, and represents the time of ventricular de-polarization and ventricular re-polarization.

ECGs are reflective of various aspects of the physical condition of the human heart and are employed, for example, to measure the rate and regularity of heartbeats, to detect the presence of damage to the heart, to monitor the effects of drugs, and for providing operating information to devices used to regulate heartbeats (e.g., defibrillators). As described briefly above, T-wave alternans, in particular, have been recognized as a significant indicator of risk for ventricular arrhythmia and sudden death. T-wave alternans result from different rates of repolarization of the muscle cells of the ventricles. The extent to which these cells nonuniformly recover or repolarize is the recognized basis for electrical instability of the heart.

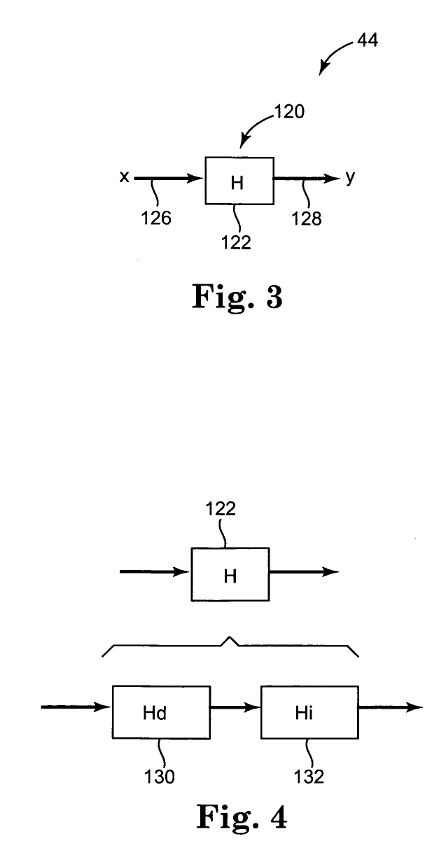

FIGS. 3-7 describe embodiments of a KR-wavelet generator 44 which is suitable for use with the wavelet matching techniques of the present invention to accurately and consistently measure T-wave alternans and other T-wave related parameters such as, for example, T-T intervals. KR-wavelet generator 44, as described below, may also be employed to measure other timing parameters such as, for example, Q-T, P-R, and P-P intervals.

With regard to FIGS. 3-7, in terms of notation, capital letters used for the transfer functions signify frequency-domain transfer functions, but imply their time-domain counterparts as well. The converse also holds true (i.e., lower-case letters signifying the impulse responses of the blocks also imply their corresponding frequency-domain transfer-function counterparts). Additionally, cascading of blocks implies multiplication of their corresponding transfer functions in the frequency domain and/or convolution of their corresponding impulse responses in the time domain, as is well established and understood in the art. (See Discrete-Time Signal Processing, by A. V. Oppenheim, R. W. Schafer, and J. R. Buck, Prentice-Hall, 1999.) Blocks are considered to be linear, time-invariant systems, and so in a given implementation the blocks include a certain cascade of blocks may be rearranged in any order within the cascade. The below-described algorithms utilized in these filter banks, may be implemented in software and its associated hardware platform, embedded circuits (hardware), firmware, or combinations thereof.

### Component Filter

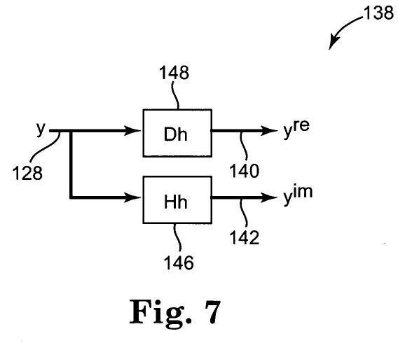

FIG. 3 is a block diagram illustrating generally a filter section 120 representative of one implementation of a KR-wavelet generator 44 formed by a component filter having a transfer function denoted by H 122. In response to a unit impulse applied at input x 126, filter section 120 provides an impulse response at output y 128. As will be described in greater detail below, this impulse response, h, is employed to generate a prototype wavelet, hₚ (See also FIG. 7 and associated discussion). In one embodiment, as illustrated by FIG. 4, transfer function H 122 is constructed from a cascade of two major blocks: a derivative kernel block Hd 130 and an integrator kernel block Hi 132.

### Scaled Derivative Kernel

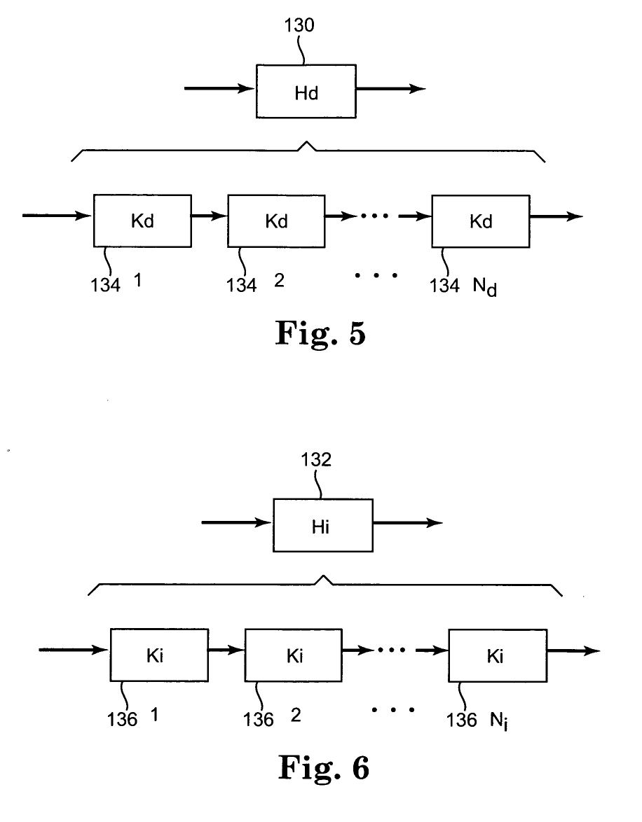

In one embodiment, as illustrated generally by FIG. 5, scaled derivative kernel block Hd 130 is formed from a cascade of N_{d} basic derivative kernel blocks Kd 134. The transfer function Kd 134 generally implements an approximation of the time derivative. Although not explicitly illustrated, per the theory of wavelets and wavelet transforms, transfer function Kd 134 is scaled by a scaling factor, k, on the frequency scale of the frequency response Kd 134 and/or of the time scale of the impulse response kd of basic derivative kernel block Kd 134. Frequency and/or time scaling serves the purpose of tuning certain characteristics of the component filter, such as, for example, the center frequency and/or bandwidth of the resulting component filter. (See The Fractional Fourier Transform, with Applications in Optics and Signal Processing, by H. M. Ozaktas, Z. Zalevsky, and M. A. Kutay, Wiley, 2000; Wavelets and Subband Coding, M. Vetterli and J. Kovacevic, Prentice-Hall, 1995).

An example embodiment of basic derivative kernel block Kd 134 is given by the discrete-time impulse response of Equation I below:

kd(n)=(δ(n)−δ(n−k))/2. Equation I:

In Equation I, the factor 2 is a normalization factor. In this example, the factor 2 is chosen to provide a unity-gain passband, but the normalization factor may be any arbitrary factor. In Equation I, δ(n) is a discrete-time delta function, sometimes referred to as the unit delta or unit impulse, which is given by Equation II below:

$$δ ( n ) = \{ \begin{matrix}1 , & n = 0 \\ 0 , & n \neq 0\end{matrix} \qquad Equation II$$

In some embodiments, in addition to delay operations, kd comprises of the operations of signed addition and scaling by a factor of two which, in some embodiments, enables implementation using a multiplierless architecture. Specifically, it is well understood in the art of digital-signal-processing architectures, that scaling a signal sample by any integer power P of 2 may be readily implemented in digital form with appropriate shifting by P bits the digital bit representation of the signal sample. Thus, by extension through the cascade, scaled-derivative kernel block Hd 130 may itself, in some embodiments, be implemented using a multiplierless architecture.

Further, the time-domain impulse response hd of scaled-derivative kernel block Hd 130 has generally a bell-shaped envelope, particularly evident for N_{d} greater than or equal to about 4, with alternating-sign coefficients. All nonzero coefficients of hd have (k-1) zeros interpolated between them. Even values of N_{d} result in hd symmetric about their time centers, while odd values of N_{d} result in hd antisymmetric about time their time centers. The intrinsic delay of the impulse response hd, equivalent to the time delay to the time center of hd, is Dd=N_{d}k/2 samples and this delay is integer-valued for N_{d} and/or k even. The z-domain transfer function of scaled-derivative kernel block Hd 130 is given by Equation III below:

$$Hd ( z ) = {[ \frac{1 - {z}^{- k}}{2} ]}^{{N}_{d}} . \qquad Equation III$$

When evaluated on the unit circle (z=e^{jω}), the frequency response of scaled derivative kernel block Hd 130 is given by Equation IV below:

$$Hd ( ω ) = {[ \frac{1 - {e}^{- jω k}}{2} ]}^{{N}_{d}} . \qquad Equation IV$$

The frequency response Hd(ω) exhibits a series of substantially bell-shaped passbands, particularly for N_{d} greater than or equal to about 4, centered at ω_{cq}=π(1+2q)/k radians, or f_{cq}=Fs(1+2q)/(2k) Hz, where Fs is the sample rate in Hz, and where q=0,1,2, . . . , such that the q=0 case corresponds to the fundamental passband centered at ω_{c0}≡ω_{c} (f_{c0}≡f_{c}). The passbands have bandwidths that are inversely proportional to scaling factor k and monotonically inversely related to the number, N_{d}, of basic derivative kernel blocks Kd 134. In the fundamental passband, the frequency response has constant (“flat”) phase equal to N_{d}π/2, after accounting for the linear phase term due to the intrinsic delay Dd.

### Scaled Integral Kernel Block

In one embodiment, as illustrated by FIG. 6, integrator kernel block Hi 132 is constructed from a cascade of Nᵢ basic integrator kernel blocks Ki 136. The transfer function Ki 136 generally implements an approximation of the local time integral (i.e., the time integral over a local interval of time). Similar to that described above with regard to transfer function Kd 134, the transfer function Ki 136 is scaled by a scaling factor, w, on the frequency scale of frequency response Ki 136 and/or on the time scale of the impulse response ki of basic integrator kernel Ki 136. An example embodiment of block Ki 136 is given by the following discrete-time impulse response of Equation V below:

ki(n)=(u(n)−u(n−w))/w. Equation V:

In Equation V, normalization by the factor w is a choice for this example to provide a unity-gain passband, but can be any arbitrary factor. Also, u(n) defines a discrete-time unit step function given by Equation VI below:

$$u ( n ) = \{ \begin{matrix}1 , & n \geq 0 \\ 0 , & n < 0\end{matrix} \qquad Equation VI$$

In some embodiments, in addition to delay operations, ki is comprised of operations of signed addition and scaling by a factor of w. In other embodiments, choosing w as an integer power of 2 enables implementation of ki using a multiplierless architecture and, by extension through the cascade, enables scaled-integral kernel block hi to be implemented using a multiplierless architecture.

In some embodiments, the time-domain impulse response of basic integrator-kernel block ki is rectangular in shape, with a width of w samples and with height w⁻¹, so that the gain at dc of this block, the sum of the coefficients, is unity. One may further note that, in some embodiments, the time-domain impulse response hi of scaled-integrator kernel block Hi 132 is generally bell-shaped, particularly evident for Nᵢ greater than or equal to about 3. In practice, in some embodiments, w is set to a value of at least 2 samples, since both w=0 and w=1 are degenerate cases resulting in all-zero coefficients and a unit impulse for hi, respectively, regardless of Nᵢ. In some embodiments, impulse responses hi are symmetric about their time center regardless of Nᵢ or w. The intrinsic delay of the impulse response hi, equivalent to the time delay to the time center of hi, is Di=Nᵢ(w−1)/2 samples and this delay is integer-valued for Nᵢ even and/or w odd. The z-domain transfer function of the scaled-integral kernel block is given by Equation VII below:

$$Hi ( z ) = {[ \frac{1 - {z}^{- w}}{w ( 1 - {z}^{- 1} )} ]}^{{N}_{i}} \qquad Equation VII$$

When evaluated on the unit circle (z=e^{jω}), the frequency response is given by Equation VIII below:

$$\begin{matrix}Hi ( ω ) = {[ \frac{1 - {e}^{- jω w}}{w ( 1 - {e}^{- jω} )} ]}^{{N}_{i}} \\ = {[ \frac{sin ( ω w / 2 )}{w sin ( ω / 2 )} ]}^{{N}_{i}} {e}^{- jω {N}_{i} ( {w}_{p} - 1 ) / 2}\end{matrix} \qquad Equation VIII$$

In some embodiments, the amplitude of Hi(ω) exhibits a classic periodic-sinc-shaped lowpass response having a mainlobe centered at dc (ω=0) with a peak amplitude of unity, sidelobes of generally decreasing amplitude towards Nyquist (ω=π), and amplitude zeros (nulls) at ω=2π·r/w radians, or f=Fs·r/w Hz, where Fs is the sample rate in Hz, and where r=1,2, . . . N. The lowpass mainlobe passband has bandwidth inversely proportional to w and monotonically inversely related to Nᵢ. With increasing Nᵢ the sidelobes become progressively more attenuated and the overall amplitude response becomes progressively more bell-shaped. After accounting for the linear phase term due to the intrinsic delay Di, the phase response of Hi(ω) is flat and equal to zero radians in the lowpass mainlobe, and continues to be flat in the sidelobes, alternating between 0 and π radians every other sidelobe for odd values of Nᵢ (i.e., the phase is flat and zero radians everywhere for even values of Nᵢ).

### Application of Hd and Hi to H

In some embodiments, as illustrated with regard to FIG. 4, filter section H 122 is composed of the cascade of scaled-derivative kernel block Hd 130 and scaled-integral kernel block Hi 132, with the frequency response of filter section H 122 being the product of the frequency responses of Hd 130 and Hi 132.

In the above description of scaled-derivative kernel block Hd 130, scaling factor k and the number N_{d} of basic derivative kernel blocks Kd serve as band-tuning parameters. In practice, band-tuning parameters k and N_{d} of Hd 130 are tuned to set the center frequency and bandwidth of the fundamental passband, and by consequence, the harmonic passbands, of filter section H 120.

Similarly, in the above description of scaled-integrator kernel block Hi 132, scaling factor w and the number Nᵢ of basic integrator kernel blocks Ki serve as band-tuning parameters. In some embodiments, band-tuning parameters w and Nᵢ of Hi 132 are tuned to set the degree of suppression needed, if any, in the harmonic passbands. This is typically accomplished by disposing the width of the mainlobe of Hi 132 so that it covers the fundamental passband of Hd 130, along with the desired number of successive harmonic passbands, if any, and so that the undesired harmonic passbands reside in the region of the nulls and sidelobes of Hi 132. If no harmonic suppression is needed, integrator kernel block Hi 132 may be eliminated, or equivalently, replaced with a unit impulse (e.g., by setting w=1).

In some embodiments, only the fundamental passband is kept, and all harmonic passbands are suppressed without loss of generality (WLOG). In some embodiments, a degree of optimality in overall harmonic suppression may be obtained by placing the first null of Hi 132 as close as possible to the peak of the first harmonic passband. In some embodiments, this constraint is satisfied by the relation given by Equation IX below:

w≡Q[2/3k] Equation IX:

where the operator Q[x] quantizes x to an integer. The operator Q[•] may take the form of the ceiling, floor, or rounding operation, and, in some embodiments, in order to satisfy the requirements of a multiplierless architecture, may be made to round to the nearest integer power of 2.

### Analytic Filter Section

FIG. 7 is a block diagram illustrating generally one embodiment of an analytic filter section 138 that can employ the above described block arrangements. In the example embodiment illustrated by FIG. 7, the output y 128 of filter section 120 is processed to produce a real output component y^{re} 140 and an imaginary output component y^{im} 142. In one embodiment, output y 128 from filter section 120 is processed through a Hilbert transformer block Hh 146 to produce a substantial approximation of the imaginary output component y^{im} 142. In other embodiments, a basic derivative kernel block Kd 134 (as per FIG. 5) is used as a particular implementation for Hilbert transformer block Hh 146 to produce a substantial approximation of the imaginary component y^{im} 142.

In one embodiment, output y 128 from filter section 120 is processed through a delay element Dh 148 to form the real output component y^{re} 140. In one embodiment, delay element Dh 148 is set for a delay substantially equal to an intrinsic delay, mh, of Hilbert transformer block Hh 146 over at least the bandwidth of interest of filter section 120. This is useful for systems requiring the intrinsic delay from y 128 to real output component y^{re} 140 to be the same as that from y 128 to imaginary output component y^{im} 142. In some embodiments, delay element Dh 148 is implemented with an impulse response given by Equation X below:

dh(n)=δ(n−mh). Equation X:

In some embodiments, basic derivative kernel block Kd 134 is used for Hilbert transformer block Hh 146. In this case, the delay of block Dh 148 may be set with mh=k/2, equivalent to the intrinsic delay of the basic derivative kernel block Kd 134. This delay is integer-valued for k even.

In some embodiments, where filter section H 122 is implemented using any real-valued wavelet, analytic filter section 138 may be applied to form a complex-valued wavelet. In some embodiments, where Hilbert transformer block Hh 146 is implemented using basic derivative kernel block Kd 134, the corresponding scale parameter k is preferably set to k=Q[π/ω_{c}], where frequency ω_{c} substantially corresponds to the central frequency of filter section H 122 (i.e. the frequency at which the magnitude of the frequency response of H 122 substantially reaches a global maximum or peak).

The outputs of analytic filter section 138 may then be used to form complex analytic output y^{c} as given by Equation XI below:

y^{C}=y^{re}+jy^{im}. Equatioon XI:

As given by Equation XI, complex analytic signal y^{c} may be generally applied for spectral analysis, instantaneous frequency and amplitude measurement, and related applications well understood and established in the art of analytic signals and communications systems.

With reference to FIG. 3, transfer function h of KR-wavelet generator 44, defined from input x 126 to output y 128 of filter section 120, can be extended to an analytic transfer function h_{c} having a real component, hᵣₑ, and an imaginary component, hᵢₘ, defined respectively from input x 126 to output y^{re} 140 and from input x 126 to output y^{im} 142, as expressed by Equation XII below:

h_{c}=hᵣ+jhᵢ. Equation XII:

### Analytic transfer function h_{c} may be applied as an analytic filter in either the time or frequency domain.

In some embodiments, where the filter section is implemented using a wavelet defined in complex-valued terms (i.e. a complex wavelet such as the Complex Morlet Wavelet or Complex Gaussian Wavelet), it is noted that impulse response h is itself complex and so may be used for complex impulse response h_{c}.

In one embodiment, a real-valued morphologic transfer function, hₚ, (i.e. a prototype wavelet) is formed from complex analytic transfer function, h_{c}, as expressed by Equation XIII below:

hₚ=aᵣhᵣₑ+aᵢhᵢₘ=aᵣreal(h_{c})+aᵢimag(h_{c}); where: Equation XIII:

hₚ=prototype wavelet transfer function;

aᵣ=real coefficient parameter; and

aᵢ=imaginary coefficient parameter;

where hᵣₑ=real(h_{c}) and hᵢₘ=imag(h_{c}) respectively represent the real and imaginary components of complex transfer function, h_{c}. In one embodiment, prototype wavelet hₚ is normalized by dividing each sample of the real and imaginary components by the amplitude of the center sample of the real component. It is noted that for hᵣₑ and hᵢₘ comprising and analytic-from Kovtun-Ricci (KR) wavelet, that prototype wavelet hₚ is referred to as a Kovtun-Ricci (KR) prototype wavelet.

Real and imaginary coefficient parameters aᵣ and aᵢ respectively represent real and imaginary parts of a complex amplitude, a, expressed by Equation XV below:

a=aᵣ+jaᵢ. Equation XIV:

The relative amplitudes of real and imaginary coefficient parameters aᵣ and aᵢ determine the morphology of the prototype wavelet, with absolute complex amplitude (|a|) and complex angle (θ) parameters being respectively expressed by Equations XVI and XVII below:

$$\begin{matrix}\vert a \vert = \sqrt{{a}_{r}^{2} + {a}_{i}^{2}} ; and \\ & Equation XV \\ θ = {tan}^{- 1} ( \frac{{a}_{i}}{{a}_{r}} ) . & Equation XVI\end{matrix}$$

With regard to Equations XV and XVI above, complex amplitude |a| generally controls the amplitude of the prototype wavelet and, as will be shown in greater detail below, complex angle θ generally controls the shape (morphology) or symmetrical properties of the prototype wavelet.

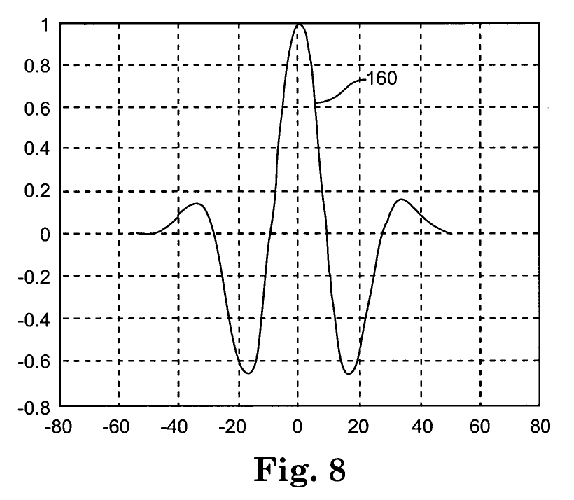

FIG. 8 is a plot 160 illustrating an example prototype wavelet generated by KR-wavelet generator 44. In the example illustration of FIG. 8, it is noted that only the real component hᵣₑ of hₚ is shown (i.e. aᵣ=1 and aᵢ=0), that the number N_{d} of basic derivative kernel blocks Kd 134 has a value of 4, and that scaling factor k has a value of 16. It is also noted that the substantially central sample of the analytic impulse response h_{c} is defined herein as the central index i_{c} of the prototype wavelet hₚ. It is noted, therefore, that the real and imaginary components of h_{c} are each centered in time around the central index i_{c}. In the illustration of FIG. 8, central index i_{c} is positioned at time equal to zero, with the example prototype wavelet being symmetric about central index i_{c}. Also, as described above, the amplitude of the prototype wavelet of FIG. 8 has been normalized so that its central value (i.e. the value at the central index) is unity.

Based on the description of KR-wavelet generator 44 with regard to FIGS. 3-7 and Equations I-XVI, the shape of the prototype wavelet hₚ can be modified or changed by adjusting the values of scaling factor k and the number N_{d} of basic derivative kernel blocks Kd 134, the values of scaling factor w and the number Nᵢ of basic integrator kernel blocks Ki 136, and the values of real coefficient parameter aᵣ and imaginary coefficient parameter aᵢ. In one embodiment, the value of the number Nᵢ of basic integrator kernel blocks Ki 136 is based on the number N_{d} of basic derivative kernel blocks Kd 134. In one embodiment, for example, the number Nᵢ of basic integrator kernel blocks comprises a ratio of the number N_{d} of basic derivative kernel blocks rounded to a nearest integer value. In one embodiment, the number Nᵢ and the number N_{d} are each set at a corresponding fixed value. In one embodiment, for example, N_{d} is set to a value of 2 and Nᵢ is set to a value of 4.

Similarly, in one embodiment, the value of scaling factor w is based on the value of scaling factor k (e.g., a ratio of scaling factor k). In one embodiment, for example, scaling factor w comprises a ratio of scaling factor k rounded (up or down) to a nearest integer value. In one embodiment, either or both values of scaling factors k and w are set at a corresponding fixed value.

As such, in one embodiment, a family of prototype wavelets can be generated by KR-wavelet generator 44 by adjusting the values of order parameters N_{d} and Nᵢ, scaling factor parameters k and w, real and imaginary coefficient parameters aᵣ and aᵢ, and central index parameter i_{c} of prototype wavelet hₚ. These parameters are sometimes referred to herein as the “wavelet parameter set” of KR-wavelet generator 44, such as illustrated by first set of wavelet parameters 46 (see FIG. 1).

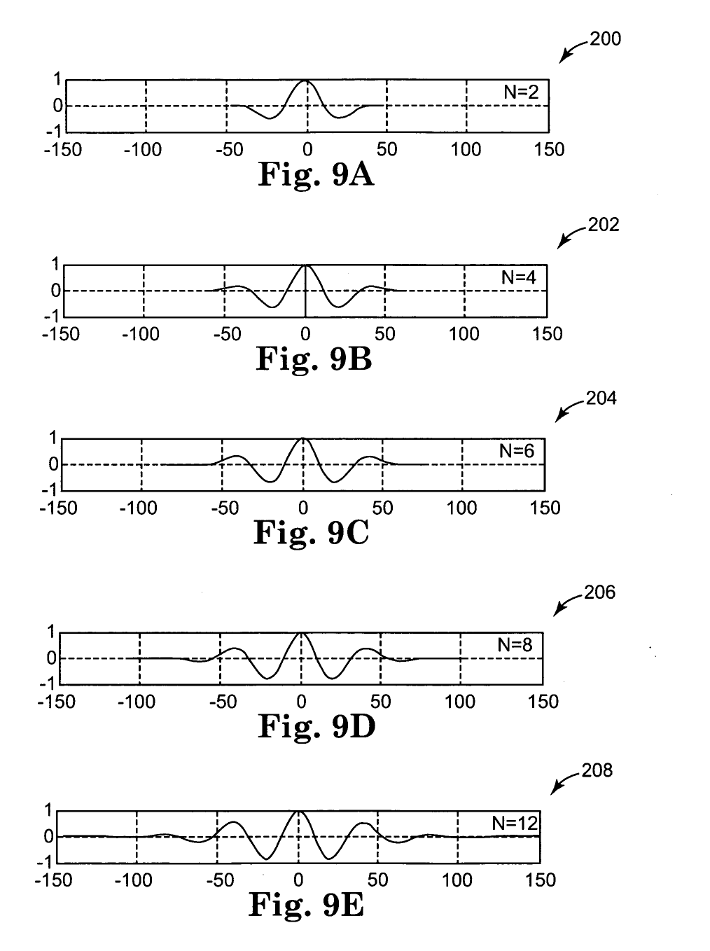

FIGS. 9A-9E respectively illustrate plots 200, 202, 204, 206, and 208 representing a subset of a family of prototype wavelets generated by KR-wavelet generator 44 and which demonstrate the effect of varying the value of order parameter N_{d} on the shape of the wavelets. Plots 200, 202, 204, 206, and 208 illustrate KR-wavelet generator 44 with a same constant scale parameter k=w, but with order parameter N_{d} respectively having values of 2, 4, 6, 8, and 12. It is noted that plots 200 through 208 illustrate only the real component hᵣₑ of hₚ (i.e. aᵣ=1 and aᵢ=0). It is also noted in plots 200, 202, 204, 206 and 208 that order parameter Nᵢ is fixed at a value of 4. As illustrated by FIGS. 9A-9E, the number of ringing cycles of each wavelet of the subset of wavelets increases with increasing values of order parameter N_{d}.

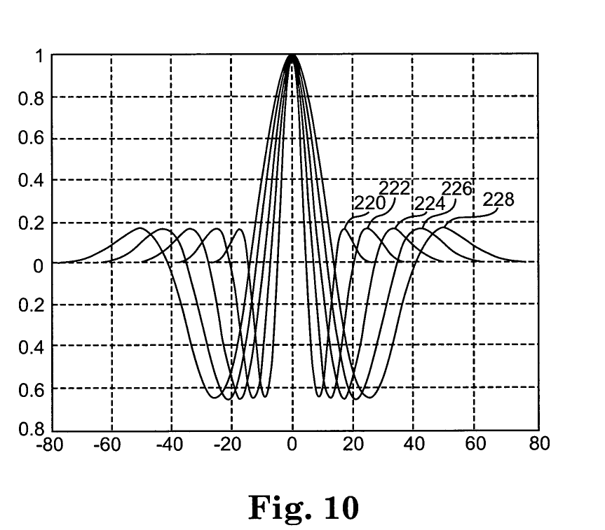

FIG. 10 illustrates plots 220, 222, 224, 226, and 228 representing a subset of a family of wavelets generated by KR-wavelet generator 44 and which demonstrate the effect of varying the value of scale parameter k on the shape of the wavelets. Plots 220, 222, 224, 226, and 228 illustrate KR-wavelet generator 44 with order parameters N_{d} and Nᵢ set to a value of 4, but with scale parameter k respectively having values of 8, 12, 16, 20, and 24. It is noted that plots 220, 222, 224, 226, and 228 illustrate only the real component hᵣₑ of hₚ (i.e. aᵣ=1 and aᵢ=0). It is also noted in plots 220, 222, 224, 226 and 228 that scale parameter w is set equal to the value of k in each case. As illustrated by FIG. 10, the widths of the prototype wavelets increase with increasing scale values.

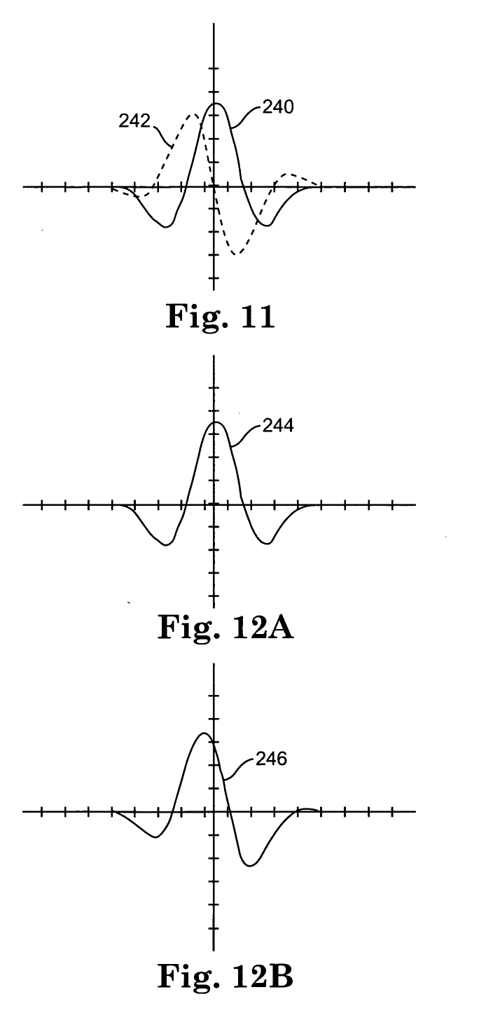

FIG. 11 illustrates plots 240 and 242 respectively representing examples of the real and imaginary components hᵣₑ and hᵢₘ of prototype wavelet hₚ with KR-wavelet generator 44 having order parameter N_{d} with a value of two (N_{d}=2). It is noted in plots 240 and 242 that order parameter Nᵢ=4, and that scale parameter w=k.

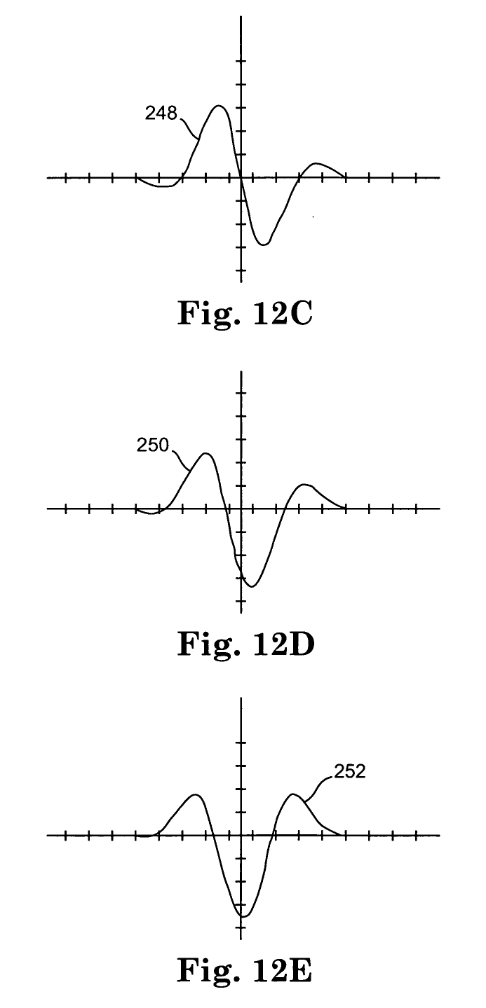

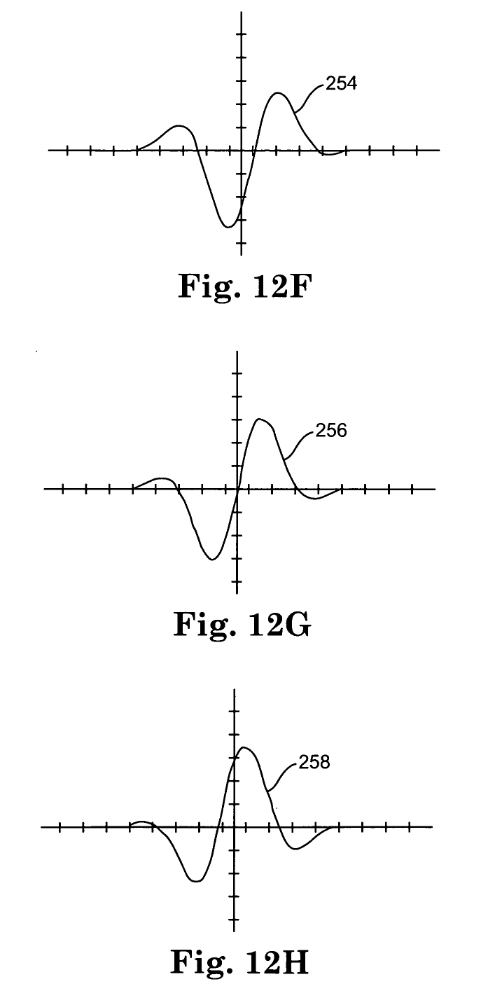

FIGS. 12A-12H respectively illustrated plots 244-258 of a subset of a family of prototype wavelets generated by KR-wavelet generator 44 which demonstrate the effect of varying the values of real and imaginary coefficient parameters aᵣ and aᵢ on the shape of the wavelets. For plots 244-258, the values of real and imaginary coefficient parameters aᵣ and aᵢ respectively correspond to particular values of complex phasor a around the complex unit circle, corresponding to complex angle θ beginning at 0 radians and incrementing by 45-degrees (π/4 radians) up to, but not including, 2π radians, such that the complex amplitude, |a|, has a value of 1 for each plot. Additionally, for each of the plots 244-258, KR-wavelet generator 44 has order parameter N_{d} having a value of two (N_{d}=2). It is noted in plots 244-258 that order parameter Nᵢ=4 and that scale parameter w=k.

Plot 244 of FIG. 12A illustrates a prototype wavelet with aᵣ=1 and aᵢ=0 (i.e. θ=0 on the unit circle). As such, plot 244 is the same as plot 240 of FIG. 11. Plot 244 represents a positive purely symmetric waveform (i.e. a positive monophasic waveform). Plot 246 of FIG. 12B illustrates a prototype wavelet with aᵣ=0.707 and aᵢ=0.707 (i.e. θ=π/4 radians on the unit circle) and represents a linear combination of a positive purely symmetric waveform (e.g., plot 244) and a positive purely anti-symmetric waveform (e.g., see FIG. 12C).

Plot 248 of FIG. 12C illustrates a prototype wavelet with aᵣ=0 and aᵢ=1 (i.e. θ=π/2 radians on the unit circle) and represents a positive purely anti-symmetric waveform (i.e. a positive biphasic waveform). Plot 250 of FIG. 12D illustrates a prototype wavelet with aᵣ=−0.707 and aᵢ=0.707 (i.e. θ=3π/4 radians on the unit circle) and represents a linear combination of a positive purely anti-symmetric waveform (e.g., plot 248) and a negative purely symmetric waveform (e.g., see FIG. 12E). Plot 252 of FIG. 12E illustrates a prototype wavelet with aᵣ=−1 and aᵢ=0 (i.e. θ=π radians on the unit circle) and represents a negative purely symmetric waveform (i.e. a negative monophasic waveform).

Plot 254 of FIG. 12F illustrates a prototype wavelet with aᵣ=−0.707 and aᵢ=−0.707 (i.e. θ=5π/4 radians on the unit circle) and represents a linear combination of a negative purely symmetric waveform (e.g., plot 252) and a negative purely anti-symmetric waveform (e.g., see FIG. 12G). Plot 256 of FIG. 12G illustrates a prototype wavelet with aᵣ=0 and aᵢ=−1 (i.e. θ=3π/2. radians on the unit circle) and represents a negative purely anti-symmetric waveform (i.e. a negative biphasic waveform). Lastly, plot 258 of FIG. 12H illustrates a prototype wavelet with aᵣ=0.707 and aᵢ=−0.707 (i.e. θ=7π/4 radians on the unit circle) and represents a linear combination of a negative purely anti-symmetric waveform (e.g., plot 256) and a positive purely symmetric waveform (e.g., see FIG. 12A).

As illustrated by FIGS. 12A-12H, by employing values of aᵣ and aᵢ over the continuum of complex angles θ between 0 and 2π, KR-wavelet generator 44 can generate prototype wavelets over a continuum of morphologies ranging from positive-to-negative and monophasic-to-biphasic and in any combination thereof. As described below in greater detail, this ability enables KR-wavelet generator 44 to generate prototype wavelets to substantially match ECG morphologies, including normal and pathologic T-wave morphologies that can be indicative of myocardial ischaemia at various stages.

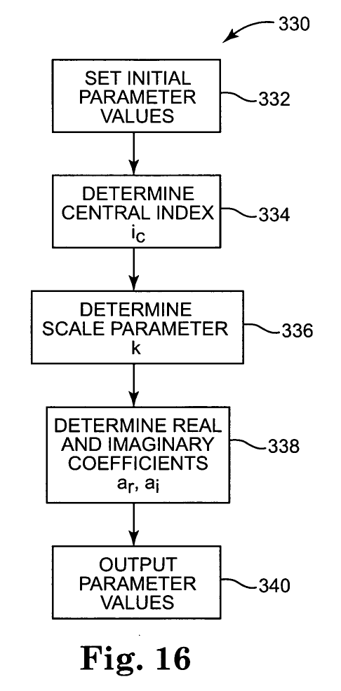

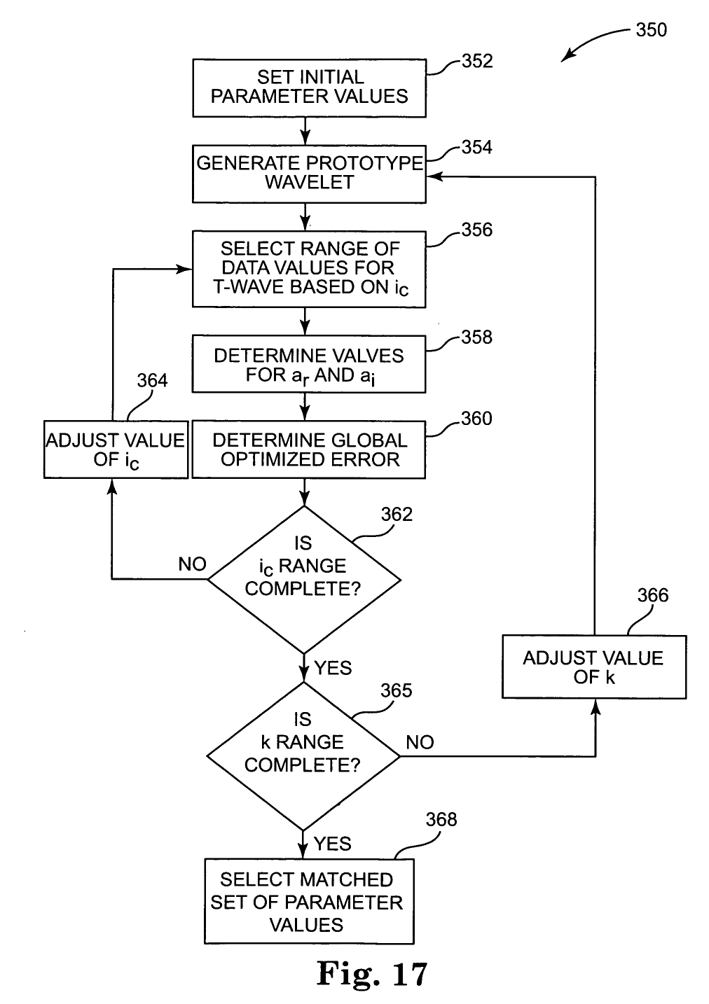

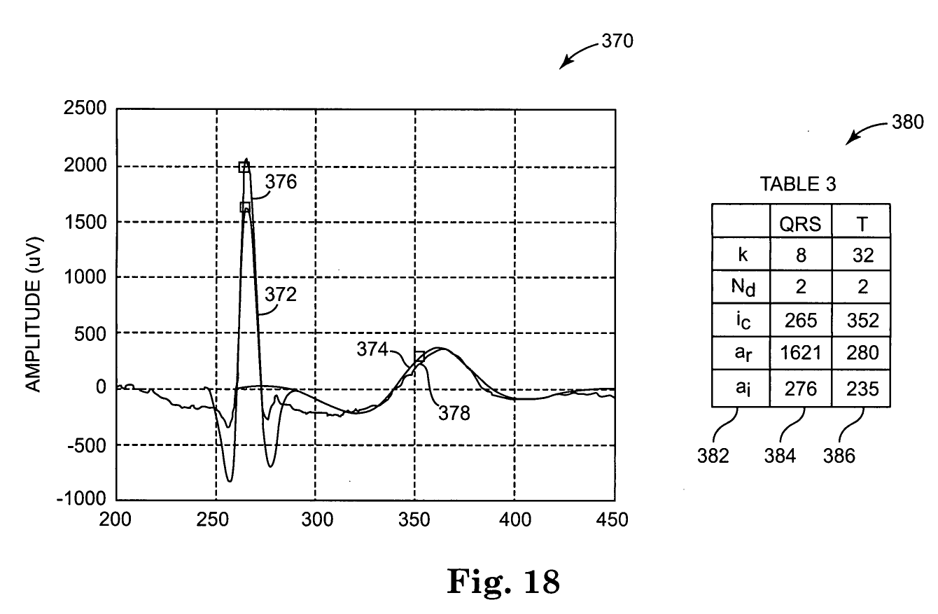

In one embodiment, input signal x 40 comprises an ECG signal with each cycle having a first feature or first target feature comprising a T-wave feature (e.g., T-wave feature 90 of FIG. 2B). In one embodiment, feature analyzer 34 of signal analyzer 30 (see FIG. 1) employs the wave-shaping characteristics described above with regard to FIGS. 9-12H beat by beat to adjust the values of the set of first wavelet parameters 46 (e.g., order parameters N_{d} and Nᵢ, scale parameters k and w, real and imaginary coefficient parameters aᵣ, and aᵢ, and central index parameter i_{c}) to adjust and match a shape of prototype wavelet hₚ generated by KR-wavelet generator 44 to each T-wave feature of a selected number of cycles of the ECG. An example of one embodiment of a matching process employed by feature analyzer is described in further detail below with regard to FIG. 16. The unique sets of matched first parameter values, each of which defines a matched prototype wavelet generated by KR-wavelet generator 44 that substantially matches a corresponding T-wave feature of the selected number of cycles of the ECG, are provided to attribute analyzer 36 via signal path 52 and employed to provide measurement of T-wave alternans and other T-wave related parameters (e.g., T-T interval).

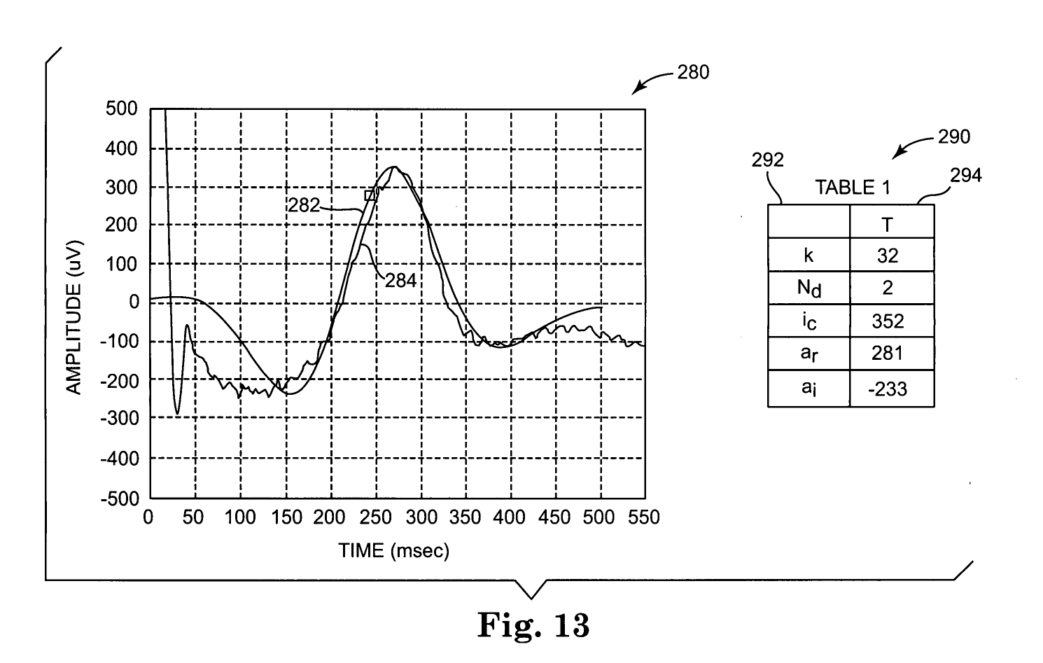

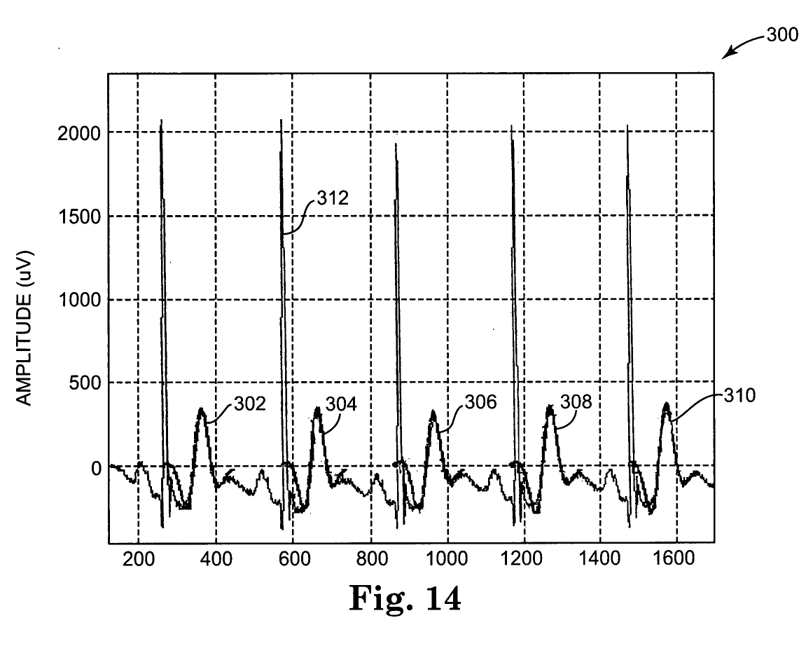

FIG. 13 is a graph 280 illustrating an example plot 282 of a matched prototype wavelet generated by KR-wavelet generator 44 after being matched to a T-wave feature 284 of an example ECG by feature analyzer 34. Table 1, indicated at 290, illustrates the matched set of first parameter values of the first set of wavelet parameters 46 corresponding to the matched prototype wavelet of plot 282. The parameters are listed in a first column 292 and their corresponding values in a second column 294. It is noted that the central index parameter i_{c} indicates a center of a support width of the prototype wavelet hₚ, and is measured relative to a time or sample index of a corresponding digital value of a selected portion of the ECG being analyzed by feature analyzer 34.

As described above, to enable measurement of T-wave alternans, feature analyzer 34 individually matches a prototype wavelet generated by KR-wavelet generator 44 to each T-wave feature of a selected number of cycles of an ECG. FIG. 14 is a graph 300 illustrating example plots 302, 304, 306, 308, and 310 of matched prototype wavelets generated by KR-wavelet generator 44 after being matched to the T-wave features of five consecutive cycles or beats of an example ECG. The spikes in the ECG signal, as illustrated by spike 312, are the R-wave feature of each cycle.

For each of the matched wavelets, feature analyzer 34 provides a set of matched first parameter values for the set of wavelet parameters 46 to attribute analyzer 36, such as those illustrated by Table 1 at 290. Attribute analyzer 36 employs this stream of sets of matched first parameter values to provide an output signal 54 indicative of T-wave related parameters, including T-wave alternans. In one embodiment, employing Equations XV and XVI, attribute analyzer 36 provides an absolute complex amplitude(|a|) parameter and a complex angle parameter θ based on real and imaginary coefficient parameters aᵣ, and aᵢ, as well as order parameter N_{d}, scale parameter k, and central index parameter i_{c}.

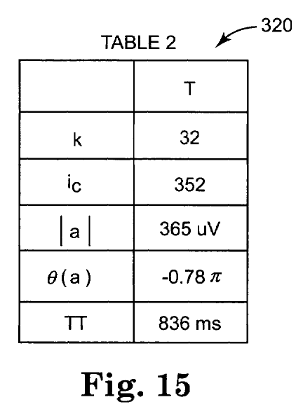

FIG. 15 is a Table 320 illustrating one example of a T-wave parameter set provided by attribute analyzer 36. It is noted that the T-wave parameters of Table 320 are based on the matched set of first parameter values of Table 290 of FIG. 13. In addition to the absolute complex amplitude(|a|) parameter and a complex angle parameter θ described above, Table 320 includes a T-T interval, which describes a time interval (in milliseconds) from the central index i_{c} of the T-wave described by the parameters of Table 320 and the corresponding central index i_{c} of an immediately preceding cycle of the ECG.

FIG. 16 is a flow diagram describing generally one embodiment of a process 330 employed by feature analyzer 34 for determining a set of optimal or matched first parameter values for the first set of wavelet parameters 46 which result in KR-wavelet generator 44 generating a prototype wavelet which substantially matches a T-wave feature of an ECG, such as T-wave 284 of FIG. 13. At 332, feature analyzer 332 sets the values of the parameters to an initial value. In one embodiment, the values may be random initial values. In one embodiment, the initial values are set based on values received from initializer 66. For example, in one embodiment, intializer 66 provides an initial value for central index parameter i_{c} based on a detected position of R-wave 86 (see FIG. 2B). In one embodiment, initializer 66 provides an initial value for scaling factor k based on an approximated magnitude of the T-wave.

Empirically, it is noted that employing an order parameter N_{d} at a value of 2 typically provides optimal matches between prototype wavelets generated by KR-wavelet generator 44 and typical T-wave features. As such, in one embodiment, as illustrated by process 330 of FIG. 16, order parameter N_{d} is set to a value of 2 and is not adjusted as part of process 330. Similarly, in one embodiment, order parameter Nᵢ is set to a value of 4 and is not adjusted as part of process 330. In other embodiments, however, the values of order parameters N_{d} and Nᵢ may be adjusted.

After setting the initial parameter values, process 330 proceeds to 334 where an optimal or matched value of central index parameter i_{c} is determined. In one embodiment, the optimal value of central index parameter i_{c} is determined by comparing a prototype wavelet generated by KR-wavelet generator 44 to a sliding window of data points of the ECG corresponding to a region in which the T-wave feature is located, wherein the sliding window has fixed width (i.e. a fixed number of data points) which matches a support width of prototype wavelet hₚ. In one embodiment, feature analyzer 34 performs a least-squares-fit between the prototype wavelet generated by KR-wavelet generator 44 and each of the data windows. The central index parameter i_{c} is determined from the data window which produces the least error from the least-squares-fit operation.

As an example, assume a given cycle of an ECG consists of 1,000 data points, with the T-wave feature being positioned approximately between data points 680-900 of the 1,000 data points, and that prototype wavelet hₚ has a support width of 200 data points. In this example, feature analyzer 34 first performs a least-squares-fit between the prototype wavelet generated by KR-wavelet generator 44 and a data window ranging between data points 680-880. Feature analyzer then performs a least-squares-fit with data points ranging between 681-881, then 681-882, and so on until ending with data points ranging between 700-900. The matched value for central index parameter i_{c} is determined from the data window which produces the least error from the least-squares-fit operation.

After determining and setting central index parameter i_{c}, process 330 proceeds to 336 where a matched value scale parameter k is determined. In one embodiment, for the range of data points of the ECG determined at 334 above to correspond most closely to the T-wave, feature analyzer 34 increments scaling factor k over a range of values and performs a least-squares-fit operation between the prototype wavelet generated by KR-wavelet generator 44 and the range of data points at each value of scaling factor k. In one embodiment, the range of values of scaling factor k ranges from a specified number of integer values below the initial value of scaling factor k to a specified number of integer values above the initial value of scaling factor k. For example, in one embodiment, the range of scaling factors k over which feature analyzer 34 performs the least squares fit operations ranges from k−1 to k+1. The value of scaling factor k which produces the least error from the least squares fit operation is selected as the matched value for scaling parameter k.

After determining and setting scaling parameter k, process 330 proceeds to 338, where feature analyzer 34 determines matched values for real and imaginary coefficient parameters aᵣ and aᵢ. In one embodiment, feature analyzer 34 increments both real and imaginary coefficient parameters aᵣ and aᵢ over ranges of values and performs a least-squares-fit operation between the range of data points determined at 334 to represent the T-wave and the prototype wavelet generated by KR-wavelet generator 44 at each of the incremental values for aᵣ and aᵢ. As above, the values for aᵣ and aᵢ resulting in the least amount of error from the least-squares-fit operations are selected as the matched values for real and imaginary coefficient parameters aᵣ and aᵢ Alternatively, in one embodiment, feature analyzer 34 employs an algorithm to directly calculate matched values for real and imaginary coefficient parameters aᵣ and aᵢ employing the matched values determined above for order parameter N, central index parameter i_{c}, and scaling parameter k.

FIG. 17 is a flow diagram describing one embodiment of an optimization process 350 employed by feature analyzer 32 for determining a set of matched first parameter values for first set of wavelet parameters 46 which define a matched wavelet that substantially matches a target feature of a waveform, such as a T-wave feature of an ECG (e.g., T-wave 284 illustrated by FIG. 13). Process 350 begins at 352, where feature analyzer 34 sets each parameter of the first set of wavelet parameters 46 to an initial value. In one embodiment, as illustrated by process 350 of FIG. 17, the values of order parameters N_{d} and Nᵢ are set to fixed values and not adjusted as part of process 350. In one embodiment, as described above with regard to 332 of process 330, N_{d} is set to a value of 2, and Nᵢ is set to a value of 4. In one embodiment, initializer 66 provides an initial value for scaling parameter k and index parameter i_{c} based on quarter-phase analysis of the ECG waveform as described by the previously incorporated U.S. patent application Ser. No. 11/360,223. In one embodiment, scaling parameter w is set equal to scaling parameter k (i.e. w=k).

At 354, based first on the initial values of the parameters provided at 352 (e.g., order parameters N_{d} and Nᵢ, and scaling parameters k and w), process 350 generates real and imaginary components hᵣₑ and hᵢₘ of prototype wavelet hₚ (see Equation XIII). At 356, feature analyzer 34 selects a segment or range of data samples of the ECG waveform representing the T-wave feature being matched, with the selected range having a width equal to the support width of prototype wavelet hₚ and centered about a data sample having a time or sample index corresponding to the central index parameter i_{c} (i.e. central sample of range based on central index parameter i_{c}).

At 358, feature analyzer 32 determines values for the real and imaginary coefficient parameters aᵣ and aᵢ of real and imaginary components hᵣₑ and hᵢₘ of prototype wavelet hₚ which optimize an error metric between prototype wavelet hₚ generated at 354 and the range of data values representing the T-wave feature selected at 356. In one embodiment, the error metric comprises a least-squares error match between prototype wavelet hₚ and the T-wave feature. In one embodiment, real and imaginary components hᵣₑ and hᵢₘ of prototype wavelet hₚ are represented in vector form and a matrix-based technique (or other linear algebraic technique such as the pseudo-inverse or singular-value decomposition, for example) is employed to directly calculate the values of real and imaginary coefficient parameters aᵣ and aᵢ.

At 360, the present optimized error from 358 (i.e. minimized error between prototype wavelet hₚ and the T-wave feature) is compared to a global optimized error based on previous iterations of process 350. When the present optimized error from 358 is less than present global optimized error, the present global optimized error is set to equal that of the present optimized error from 358 and the present global optimized error and values of the corresponding first set of wavelet parameters (e.g., N_{d}, Nᵢ, k, w, aᵣ, aᵢ, and i_{c}) are stored at a memory location. For the first iteration, it is noted that the global optimized error is set to the optimized error from 360. Process 350 then proceeds to 362 where it is queried whether aᵣ and aᵢ values and an associated optimized error have been determined with the present set of values for N_{d}, Nᵢ, k, and w for each value (e.g., sample or time index) of a range of values for central index parameter i_{c}. In one embodiment, the range of values for central index parameter i_{c} comprises a predetermined number of samples centered about the initial value of ic determined at 352. In one embodiment, the range of values for central index parameter i_{c} comprises seven (7) samples, with three of the samples immediately preceding and three immediately following the sample corresponding to the initial value of i_{c} determined at 352.

If the answer to the query at 362 is “no”, process 350 proceeds to 364 where the value of i_{c} is adjusted to a next value of the range of values for i_{c}. At 356, a new range of data samples for the T-wave features and centered about the adjusted central index parameter i_{c} is selected and 358, 360, and 362 are repeated. If the answer to the query at 362 is “yes”, process 350 proceeds to 365 where it is queried whether a prototype wavelet has been generated at 354 for each value of a range of integer values for scaling parameter k (with scaling parameter w=k for process 350).

In one embodiment, the range of values for k comprises a predetermined number of integer values centered about the initial value of scaling factor k determined at 352. In one embodiment, the range comprises three integer values ranging from k−1 to k+1, with the value of k being the initial value determined at 352. If the answer to the query at 365 is “no”, process 350 proceeds to 366 where the value of scaling parameter k is adjusted to a next value of the range of values and 354, 356, 358, 360, 362 and 364 are repeated.

If the answer to the query at 365 is “yes”, process 350 proceeds to 368 where the parameter values corresponding to the present value of the global optimized error at 360 are set as the values of the matched set of first wavelet parameters for the given T-wave feature. Process 350 is then repeated for each T-wave feature of the selected number of cycles of the ECG waveform being analyzed by signal analyzer 30. In one embodiment, the matched set of first parameter values from 368 are employed as the initial values for N_{d}, Nᵢ, k, and w for the next T-wave feature to be analyzed.

In one embodiment, as described above with regard to FIG. 1, feature analyzer 34 employs a second wavelet generator 58, the second wavelet generator 58 being configured to generate a family of prototype wavelets defined by a second set of wavelet parameters 60, to match a second feature of each cycle of input signal x 40. In one embodiment, second wavelet generator 58 comprises the same wavelet generator as first wavelet generator 44, with second set of wavelet parameters 60 being the same as first set of wavelet parameters 46. In one embodiment, as described above, input signal x 40 is an ECG with each cycle having a first feature comprising a T-wave and a second feature comprising a QRS-complex. As such, in one embodiment, in addition to matching the T-wave feature of each of the selected number of cycles of the ECG, feature analyzer 30 matches a prototype wavelet to the QRS-complex (see QRS-complex 92 of FIG. 2B) of each of the selected number of cycles.

In a fashion similar to that described above by FIGS. 13-16 with regard to T-waves, feature analyzer 34 adjusts the values of the parameters of first wavelet parameter set 46 (e.g., order parameter N_{d}, scale parameter k, real and imaginary coefficient parameters aᵣ, and aᵢ, and central index parameter i_{c}) to obtain a matched wavelet for the QRS-complex of each cycle of the selected number of cycles of an ECG. As before, the unique sets of matched first parameter values corresponding to the matched prototype wavelet of each of the QRS-complex features are provided to attribute analyzer 36 via signal path 52 and employed to provide measurement of the QRS-complex, ECG timing parameters (e.g., R-R intervals), and T-wave related parameters (e.g., R-T intervals).

FIG. 18 is a graph 370 illustrating example plots 372 and 374 of matched wavelets generated by KR-wavelet generator 44 after respectively being matched to a QRS-complex 376 and a T-wave feature 378 of an example ECG by feature analyzer 34. Table 3, indicated at 380, illustrates the set of matched first parameter values of the first set of wavelet parameters 46 corresponding to example plots 372 and 374. The parameters are listed in a first column 382, with their corresponding matched values for QRS-complex 376 and T-wave feature 378 being respectively listed in second and third columns 384 and 386.

In such an embodiment, the sets of matched first parameter values for both the QRS-complex and the T-wave features, as illustrated by Table 3 at 380 are provided to attribute analyzer 36. In one embodiment, attribute analyzer in-turn employs the sets of matched wavelet first parameter values to provide an output signal 54 (see FIG. 1) indicative of T-wave related parameters, including T-wave alternans, as well as other timing related parameters (e.g., R-T and R-R intervals).

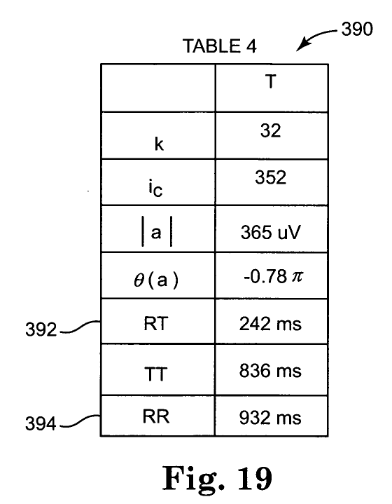

FIG. 19 is a Table 390 illustrating one example of a T-wave parameter set provided by attribute analyzer 36. It is noted, that in addition to the scaling parameter k, central index parameter i_{c}, absolute complex amplitude(|a|) parameter, complex angle parameter θ, and T-T interval described above with regard to Table 320 of FIG. 15, Table 390 further includes an R-T interval, indicated at 392, and an R-R interval, indicated at 394. R-T interval 392 describes a time interval (in milliseconds) from the central index i_{c} corresponding to T-wave 378 and the central index i_{c} corresponding to QRS-complex 376. R-R interval 394 describes a time interval (i.e. heart rate) from the central index i_{c} corresponding to QRS-complex 376 and the central index corresponding to a QRS-complex of an immediately preceding cycle of the ECG.

Although described above with regard to FIGS. 13-19 in terms of performing a single matching operation of a prototype wavelet generated by KR-wavelet generator 44 to a target feature, such as T-wave feature 284 of FIG. 13, so as to generate a matched set of first parameter values for first set of wavelet parameters 46 for determination of T-wave alternans and other T-wave related parameters, the wavelet matching techniques described herein can also be applied iteratively to a single target feature for purposes of further analysis or for purposes of data compression.

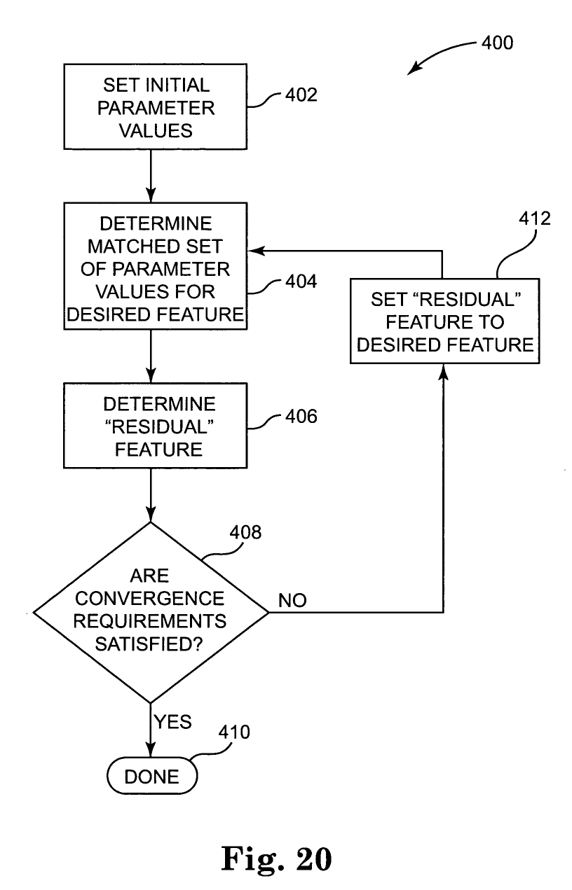

FIG. 20 is a flow diagram generally illustrating one embodiment of a process 400 employed by feature analyzer 34 for iteratively matching prototype wavelets generated by KR-wavelet generator 44 to a feature of a waveform, such as T-wave feature 284 of an ECG, for data analysis or for data compression purposes. Process 400 is described with further reference to FIGS. 13 and 16.

Process 400 begins at 402, where feature analyzer 34 sets the values of the first set of wavelet parameter set 46 to an initial value, such as described above at 332 with regard to process 330 of FIG. 16. Process 400 then proceeds to 404, where feature analyzer 34 adjusts the values of first set of wavelet parameters 46 to determine a set of matched first parameter values that results in KR-wavelet generator 44 generating a prototype wavelet that substantially matches a target feature. At 404, in one embodiment, feature analyzer 36 employs a matching process similar to that described above by 334-340 of process 330 of FIG. 16.

Process 400 then proceeds to 406, where the matched wavelet generated at 404 is subtracted from the data values representing the target feature to create a residual feature. Process 400 then proceeds to 408, where it is queried whether a convergence requirement has been satisfied. In one embodiment, the convergence requirement is satisfied when an amplitude of the residual feature is less than or equal to a desired level, such that the target feature has been effectively removed from the ECG. In one embodiment, the convergence requirement is satisfied when 404, 406, and 412 (as described below) have been repeated, or iterated, a predetermined number of times.

If the answer to the query at 408 is “yes”, process 400 is completed, as indicated at 410. If the answer to the query at 408 is “no”, process 400 proceeds to 412 where the target feature is set to the residual feature. Another set of matched first parameter values is then determined at 404 to again generate a prototype wavelet which substantially matches the target feature (i.e. the present residual feature), and 406 and 408 are repeated accordingly.

In one embodiment, the series of the sets of matched first parameter values for the first set of wavelet parameters 46 determined at 404 are provided to attribute analyzer 36 for subsequent storage as wavelet parameter objects (see FIG. 1) in a storage device, such as storage device 56. The wavelet parameter objects can be grouped according to their order of iteration, and the T-wave feature may be reconstructed by summing the matched prototype wavelets generated by KR-wavelet generator 44 corresponding to the series of sets of matched first parameter values.

It is noted that process 400 can be applied to any number of desired features of a waveform so that the waveform can be stored and transmitted as a series of wavelet parameter objects in lieu of the original series of data points, such as input signal x 40, thereby reducing storage and transmission bandwidth requirements.

Although described herein in detail with respect to matching the QRS-complex and T-wave features of ECG, the wavelet matching techniques described herein can be adapted to apply to and measure parameters of other ECG features, such as, for example, S-T segments and parameters associated with P-waves. For example, although not illustrated, in one embodiment, the above described wavelet matching techniques can be employed to determine a matched wavelet for each P-wave feature of the selected number of cycles of the ECG. The central index parameter i_{c} corresponding to each P-wave feature can then be employed to determine a P-P interval between consecutive P-wave features and, together with the central index parameter i_{c} corresponding to each QRS-complex feature, be employed to determine a P-R interval between the P-wave and QRS-complex feature of each cycle of the selected number of cycles.

Furthermore, the wavelet matching techniques described herein can also be adapted to be employed with other types of waveforms or signals, such as electroencephalograms (EEGs), electromyograms (EMGs), and electro-olfactograms (EOGs), and other signals having sufficiently time-separated features. Additionally, although described in detail with respect to a Kovtun-Ricci wavelet, other wavelets having suitable characteristics may be employed to implement the wavelet matching techniques described herein.

Although specific embodiments have been illustrated and described herein, it will be appreciated by those of ordinary skill in the art that a variety of alternate and/or equivalent implementations may be substituted for the specific embodiments shown and described without departing from the scope of the present invention. This application is intended to cover any adaptations or variations of the specific embodiments discussed herein. Therefore, it is intended that this invention be limited only by the claims and the equivalents thereof.

What is claimed is:

1. A method of analyzing an elecotrocardiogram (ECG) waveform, the method comprising:
obtaining a series of digital values representing an ECG waveform having a plurality of cycles, each cycle having a T-wave feature;
comparing a first prototype wavelet to a portion of the series of digital values representing at least the T-wave feature of a selected number of cycles of the ECG waveform, wherein the first prototype wavelet is defined by a first set of wavelet parameters; and
determining a matched set of first parameter values for the first set of wavelet parameters which define a matched wavelet for each of the T-wave features of the selected number of cycles, wherein each matched wavelet substantially matches the corresponding T-wave feature.

2. The method of claim 1, wherein determining the matched set of first parameter values includes:
setting the first set of wavelet parameters to an initial set of first parameter values; and
adjusting the initial set of first parameter values to obtain the matched set of first parameter values for each of the T-wave features.

3. The method of claim 2, wherein adjusting the initial set of first parameter values to obtain the set of matched first parameter values for each of the T-wave features includes substantially optimizing an error metric indicative of a closeness of the match between the first prototype wavelet and the T-wave feature.

4. The method of claim 3, wherein the error metric comprises a least-squares-fit between the first prototype wavelet and the T-wave feature.

5. The method of claim 1, comprising:
generating the first prototype wavelet.

6. The method of claim 1, comprising:
providing an output indicative of T-wave alternans of the T-wave features of the selected number of cycles based on the corresponding sets of matched first parameter values.

7. The method of claim 1, wherein the first prototype wavelet comprises a real-valued wavelet formed from the linear combination of a real-part wavelet component and an imaginary-part wavelet component of a complex-valued analytic wavelet, and wherein the first set of wavelet parameters includes a real coefficient parameter associated with the real-part wavelet component and an imaginary coefficient parameter associated with the imaginary-part wavelet component of the complex-valued analytic wavelet.

8. The method of claim 7, wherein the complex-valued analytic wavelet is formed from a real-valued wavelet, and wherein a time-adjusted form of the real-valued wavelet is used to form the real-part wavelet component of the complex-valued analytic wavelet, and a Hilbert transformer is applied to the real-valued wavelet to from the imaginary-part wavelet component of the complex-valued analytic wavelet.

9. The method of claim 7, wherein the complex-valued wavelet is formed from a real-valued wavelet, and wherein a time-adjusted form of the real-valued wavelet is used to from the real-part wavelet component of the complex-valued analytic wavelet, and an approximation of a Hilbert transformer is applied to the real-valued wavelet to form the imaginary-part wavelet component of the complex-valued analytic wavelet.

10. The method of claim 7, comprising:
determining an absolute complex amplitude for each T-wave feature of the selected number of cycles based on the values of the real and imaginary coefficient parameters of the corresponding set of matched first parameter values.

11. The method of claim 7, comprising:
determining a complex angle for each T-wave feature of the selected number of cycles based on the values of the real and imaginary coefficient parameters of the corresponding set of matched first parameter values.

12. The method of claim 1, wherein the first set of wavelet parameters includes a central index parameter indicative of a time position of each T-wave feature of the selected number of cycles, wherein the time position is relative to a beginning of the selected number of cycles.

13. The method of claim 12, comprising:
providing an output indicative of a T-T interval between consecutive T-wave features of the selected number of cycles based on the values of the central index parameters of the corresponding sets of matched first parameter values.

14. The method of claim 1, wherein the set of first wavelet parameters includes at least one order parameter and at least one scale parameter.

15. The method of claim 1, wherein the first prototype wavelet comprises a Kovtun-Ricci wavelet.

16. The method of claim 1, wherein each cycle of the ECG waveform includes a QRS complex feature, and wherein the method comprises:
comparing a second prototype wavelet to the portion of the series of digital values representing at least the QRS complex of the selected number of cycles of the ECG waveform, wherein the second prototype wavelet is defined by a second set of wavelet parameters; and
determining a matched set of second parameter values for the second set of wavelet parameters which define a matched wavelet for each of the QRS complex features of the selected number of cycles, wherein each matched wavelet substantially matches the corresponding QRS complex feature.

17. The method of claim 16, wherein the second prototype wavelet is the same as the first prototype wavelet and the second set of wavelet parameters comprises the same parameters as the first set of wavelet parameters.

18. The method of claim 16, wherein the second set of wavelet parameters includes a central index parameter indicative of a time position of each QRS-complex feature of the selected number of cycles, wherein the time position is relative to a beginning of the selected number of cycles.

19. The method of claim 16, comprising:
providing an output indicative of an R-R interval between consecutive R-wave features of the selected number of cycles based on the values of the central index parameters of the corresponding sets of matched second parameter values.

20. The method of claim 16, comprising:
providing an output indicative of an R-T interval between the QRS-complex feature and the T-wave feature of a same cycle for each of the selected number of cycles based on the corresponding sets of matched first and second parameter values.

21. A signal analyzer comprising:
a receiver configured to obtain a series of digital values representing an ECG waveform having a plurality of cycles, each cycle having at least a T-wave feature; and
a feature analyzer configured to compare a first prototype wavelet defined by a first set of wavelet parameters to a portion of the series of digital values representing at least the T-wave feature of a selected number of cycles of the ECG waveform, and configured to determine a matched set of first parameter values for the first set of wavelet parameters which define a matched wavelet for each of the T-wave features of the selected number of cycles, wherein each matched wavelet substantially matches the corresponding T-wave feature.

22. The signal analyzer of claim 21, comprising:
an initializer configured to set the first set of wavelet parameters to an initial set of first parameter values, and wherein the feature analyzer is configured to adjust the initial set of first parameter values to obtain the matched set of first parameter values for each of the T-wave features.

23. The signal analyzer of claim 22, wherein the feature analyzer is configured to adjust the initial set of first parameter values to substantially optimize an error metric indicative of a closeness of a match between the first prototype wavelet and the T-wave feature.

24. The signal analyzer of claim 21, wherein the feature analyzer is configured to generate the first prototype wavelet.

25. The signal analyzer of claim 21, wherein the first prototype wavelet comprises a Kovtun-Ricci wavelet.

26. The signal analyzer of claim 21, wherein the first prototype wavelet comprises a real-valued wavelet formed from the linear combination of a real-part wavelet component and an imaginary-part wavelet component of a complex-valued analytic wavelet, and wherein the first set of wavelet parameters includes a real coefficient parameter associated with the real-part wavelet component and an imaginary coefficient parameter associated with the imaginary-part wavelet component of the complex-valued analytic wavelet.

27. The signal analyzer of claim 26, wherein the complex-valued analytic wavelet is formed from a real-valued wavelet, and wherein a time-adjusted form of the real-valued wavelet is used to form the real-part wavelet component of the complex-valued analytic wavelet, and a Hilbert transformer is applied to the real-valued wavelet to from the imaginary-part wavelet component of the complex-valued analytic wavelet.

28. The signal analyzer of claim 26, wherein the complex-valued wavelet is formed from a real-valued wavelet, and wherein a time-adjusted form of the real-valued wavelet is used to from the real-part wavelet component of the complex-valued analytic wavelet, and an approximation of a Hilbert transformer is applied to the real-valued wavelet to form the imaginary-part wavelet component of the complex-valued analytic wavelet.

29. The signal analyzer of claim 21, comprising:
an attribute analyzer configured to determine at least one characteristic of the T-wave features of the selected number of cycles based on the corresponding sets of matched first parameter values.

30. The signal analyzer of claim 29, wherein the at least one characteristic includes an absolute complex amplitude of each T-wave.

31. The signal analyzer of claim 29, wherein the at least one characteristic includes a complex angle of each T-wave.

32. The signal analyzer of claim 21, wherein the first set of wavelet parameters includes a central index parameter indicative of a time position of each T-wave feature of the selected number of cycles, wherein the time position is relative to a beginning of the selected number of cycles.

33. The signal analyzer of claim 23, wherein an attribute analyzer is configured to provide an output indicative of a T-T interval between consecutive T-wave features of the selected number of cycles based on the values of the central index parameters of the corresponding sets of matched first parameter values.

34. The signal analyzer of claim 18, wherein each cycle of the ECG waveform includes a QRS complex feature, and wherein the feature analyzer is configured to compare a second prototype wavelet defined by a second set of wavelet parameters to the portion of the series of digital values representing the selected number of cycles of the ECG waveform, and configured to determine a matched set of second parameter values for each of the QRS-complex features of the selected number of cycles, wherein each matched wavelet substantially matches the corresponding QRS-complex feature.

35. The signal analyzer of claim 34, wherein the second prototype wavelet is the same as the first wavelet and the second set of wavelet parameters comprises the same parameters as the first set of wavelet parameters.

36. The signal analyzer of claim 34, wherein an attribute analyzer is configured to provide an output indicative of an R-R interval between consecutive QRS-complex features of the selected number of cycles based on the values of the corresponding sets of matched second parameter values.

37. The signal analyzer of claim 34, wherein an attribute analyzer is configured to provide an output indicative of an R-T interval between the QRS-complex feature and the T-wave feature of a same cycle for each of the selected number of cycles based on the corresponding sets of matched first and second parameter values.

38. A signal analyzer comprising:
means for obtaining a series of digital values representing an ECG waveform having a plurality of cycles, each cycle having a T-wave feature;
means for comparing a first prototype wavelet to a portion of the series of digital values representing at least the T-wave feature of a selected number of cycles of the ECG waveform, wherein the first prototype wavelet is defined by a first set of wavelet parameters; and
means for determining a matched set of first parameter values for the first set of wavelet parameters for each of the T-wave features of the selected number of cycles, wherein each matched wavelet substantially matches the corresponding T-wave feature.

39. The signal analyzer of claim 38, wherein the means for determining includes:
means for setting the first set of wavelet parameters to an initial set of first parameter values; and
means for adjusting the initial set of first parameter values for each of the T-wave features.

40. The signal analyzer of claim 39, wherein the means for adjusting includes means for determining and substantially optimizing an error metric indicative of a closeness of a match between the first prototype wavelet and the T-wave feature.

41. The signal analyzer of claim 38, comprising:
means for determining at least one characteristic of the T-wave features of the selected number of cycles based on the corresponding sets of matched first parameter values, wherein the at least one characteristic includes T-wave alternans.

42. A method of analyzing an elecotrocardiogram (ECG) waveform, the method comprising:
obtaining a series of digital values representing an ECG waveform having a plurality of cycles, each cycle having a P-wave feature;
comparing a first prototype wavelet to a portion of the series of digital values representing at least the P-wave feature of a selected number of cycles of the ECG waveform, wherein the first prototype wavelet is defined by a first set of wavelet parameters; and
determining a matched set of first parameter values for the first set of wavelet parameters which define a matched wavelet for each of the P-wave features of the selected number of cycles, wherein each matched wavelet substantially matches the corresponding P-wave feature.

43. The method of claim 42, wherein the first set of wavelet parameters includes a central index parameter indicative of a time position of each P-wave feature of the selected number of cycles, wherein the time position is relative to a beginning of the selected number of cycles, and wherein the method comprises:
providing an output indicative of a P-P interval between consecutive P-wave features of the selected number of cycles based on the central index parameters of the corresponding sets of matched first parameter values.

44. The method of claim 43, wherein each cycle of the ECG includes a QRS-complex, and wherein the method comprises:
comparing a second prototype wavelet to the portion of the series of digital values representing at least the QRS complex of the selected number of cycles of the ECG waveform, wherein the second prototype wavelet is defined by a second set of wavelet parameters; and
determining a matched set of second parameter values for the second set of wavelet parameters which define a matched wavelet for each of the QRS complex features of the selected number of cycles, wherein each matched wavelet substantially matches the corresponding QRS complex feature.

45. The method of claim 43, wherein the second set of wavelet parameters includes a central index parameter indicative of a time position of each QRS-complex feature of the selected number of cycles, wherein the time position is relative to the beginning of the selected number of cycles, and wherein the method comprises:
providing a P-R interval between the P-wave and QRS-complex features of a same cycle for each of the selected number of cycles based on the corresponding sets of matched first and second parameter values.
